(19) United States
(12) Patent Application Publication
(10) Pub. No.: US 2024/0100306 A1
(43) Pub. Date: Mar. 28, 2024

(54) Instrument Delivery Device with Antithrombogenic Features

(71) Applicant: Becton, Dickinson and Company, Franklin Lakes, NJ (US)

(72) Inventors: Shelby Ann Bieritz, Durham, NC (US); Yiping Ma, Layton, UT (US)

(21) Appl. No.: 18/371,194

(22) Filed: Sep. 21, 2023

Related U.S. Application Data
(60) Provisional application No. 63/408,973, filed on Sep. 22, 2022.

(51) Int. Cl. A61M 25/09; A61M 25/00; A61M 25/06

(52) U.S. Cl. CPC A61M 25/09041; A61M 25/0045; A61M 25/0097; A61M 25/0606; A61M 2025/09066

(57) ABSTRACT

Provided herein is a device for advancement of an instrument into an intravenous catheter assembly. The device includes a housing having a proximal end, a distal end, and a sidewall therebetween defining an interior. A displaceable instrument is received within the housing interior and has a proximal end and a distal end, with the instrument being one of a guidewire and a probe. The instrument is advanceable from a first, proximal position in which the distal end does not extend beyond a distal end of a catheter of the intravenous catheter assembly, to a second, distal position in which the distal end extends beyond the distal end of the catheter of the intravenous catheter assembly. The distal end of the instrument includes an antithrombogenic material, the antithrombogenic material being an antithrombogenic coating applied to the instrument or an antithrombogenic composition from which the instrument is formed, in part.

### CROSS-REFERENCE TO RELATED APPLICATION

The present application claims priority to U.S. Provisional Application Ser. No. 63/408,973, entitled “Instrument Delivery Device with Antithrombogenic Features” filed Sep. 22, 2022, the entire disclosure of which is hereby incorporated by reference in its entirety.

### BACKGROUND OF THE INVENTION

### Field of the Invention

Provided herein are devices and systems for use in vascular access, and, in particular, devices and systems for introducing an instrument through indwelling peripheral catheters.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for blood draw or introducing an instrument for diagnostic testing/monitoring. Among these limitations are that, the smaller gauge of the instrument that is necessary to pass through the PIVC to the patient's vasculature and/or the long dwell time of the instrument in the vasculature (e.g., during blood draw or prolonged diagnostic testing) can result in thrombogenesis and failure of the blood collection or testing procedure. Accordingly, there is a need in the art for devices and systems that allow for the introduction of instruments into a PIVC with reduced risk of thrombogenesis.

### SUMMARY OF THE INVENTION

Provided herein is an instrument delivery device for advancement of an instrument into an intravenous catheter assembly. The instrument delivery device includes a housing having a proximal end, a distal end, a sidewall therebetween defining an interior, and a connecter at the distal end of the housing for reversibly connecting the instrument delivery device to the intravenous catheter assembly. A displaceable instrument is received within the housing interior and has a proximal end and a distal end, with the instrument being one of a guidewire and a probe. The instrument is advanceable from a first, proximal position in which the distal end does not extend beyond a distal end of a catheter of the intravenous catheter assembly, to a second, distal position in which the distal end extends beyond the distal end of the catheter of the intravenous catheter assembly. The distal end of the instrument includes an antithrombogenic material, the antithrombogenic material being an antithrombogenic coating applied to the instrument or an antithrombogenic composition from which the instrument is formed, in part.

In certain configurations, where the antithrombogenic material is an antithrombogenic coating, the antithrombogenic coating includes one or more of a hydrophilic material, heparin, a heparin mimetic, albumin, a hydrophilic lubricant, a fluorinated lubricant, and a triblock polymer.

In certain configurations, the triblock polymer comprises poly(ethylene oxide) (PEO) and/or poly(propylene, oxide) (PPO).

In certain configurations, the triblock polymer is a PEO-PPO-PEO polymer.

In certain configurations, where the antithrombogenic material is an antithrombogenic coating, the antithrombogenic coating includes poly(ethylene glycol) or poly(ethylene oxide), one or more sulfate and/or sulfonate groups, and heparin.

In certain configurations, where the antithrombogenic material is an antithrombogenic coating, the antithrombogenic coating includes any of phosphorylcholine and poly(2-methoxyethyl acrylate).

In certain configurations, where the antithrombogenic material is an antithrombogenic composition, the antithrombogenic composition includes fluoro or fluorine-containing functional moieties.

In certain configurations, where the antithrombogenic material is an antithrombogenic composition, the antithrombogenic composition includes an antibiotic or antiseptic comprising minocycline-rifampicin or chlorhexidine-silver sulphadiazine.

In certain configurations, the instrument is a guidewire that includes a core wire and a coil member wrapped around the core wire, at a distal end of the core wire, with the antithrombogenic material provided in or on the distal end of the core wire and the coil member.

In certain configurations, the instrument is a coreless coiled guidewire, with the antithrombogenic material provided in or on a distal end of the coreless coiled guidewire

In certain configurations, the instrument is a probe that includes a support wire and a sensor positioned at a distal end of the support wire configured to measure one or more physiological parameters, with the antithrombogenic material provided in or on the distal end of the support wire and applied to the sensor as an antithrombogenic coating.

In certain configurations, the instrument is a probe that includes an outer tubing, an optical fiber contained within the outer tubing, and a sensor coupled to a distal end of the optical fiber, at a distal tip of the outer tubing, with the sensor configured to measure one or more physiological parameters. The antithrombogenic material is provided in or on the distal tip of the outer tubing and applied to the sensor as an antithrombogenic coating.

In certain configurations, the sensor is configured to acquire measurements of the one or more physiological parameters over a continuous period of time.

In certain aspects, application of the antithrombogenic coating to the sensor prevents fouling of the sensor.

In certain configurations, the antithrombogenic material is provided in or on an entire length of the instrument.

In certain configurations, an advancement member is positioned on the housing and is moveable relative thereto, with the advancement member coupled to the instrument such that movement of the advancement member causes a corresponding movement of the instrument.

In certain configurations, the connector comprises a plurality of arms configured to engage a needleless connector.

In certain configurations, the connector includes a blunted cannula configured to pierce a septum of the needleless connector.

Also provided herein is a catheter system, including a catheter assembly including a catheter adapter having a distal end, a proximal end, a lumen extending between the distal end and the proximal end, and a catheter secured to the catheter adapter and extending distally from the catheter adapter, wherein the catheter has a distal end and a proximal end, and an instrument delivery device as described herein.

In certain configurations, the catheter assembly includes a needleless access connector, wherein the distal end of the instrument delivery device housing is reversibly coupleable to the needleless access connector.

### BRIEF DESCRIPTION OF THE DRAWINGS

FIG. 1 is a perspective view of a non-limiting embodiment of a prior art catheter assembly useful with instrument delivery devices as described herein;

FIGS. 2A and 2B are side views of a non-limiting embodiment of an instrument delivery device as described herein;

FIG. 3A is a partial cross-sectional side view of an instrument useable with the instrument delivery device of FIGS. 2A and 2B, in accordance with one aspect of the disclosure;

FIG. 3B is a partial cross-sectional side view of an instrument useable with the instrument delivery device of FIGS. 2A and 2B, in accordance with another aspect of the disclosure;

FIG. 3C is a partial cross-sectional side view of an instrument useable with the instrument delivery device of FIGS. 2A and 2B, in accordance with another aspect of the disclosure;

FIG. 3D is a partial cross-sectional side view of an instrument useable with the instrument delivery device of FIGS. 2A and 2B, in accordance with another aspect of the disclosure; and

FIG. 4 is a perspective view of a non-limiting embodiment of a system including an instrument delivery device as described herein.

### DETAILED DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein are devices and systems for introducing instruments through indwelling catheters, such as peripheral intravenous catheters (PIVC s). While certain instrument delivery devices are shown in the accompanying figures and described below, such as in the context of aiding in performing a blood draw, those of skill will appreciate that antithrombogenic features described herein may be useful in any number of different devices and applications for introducing an instrument, including instruments ranging from tubes, probes, sensors, wiring, fiber optics, guidewires, etc.

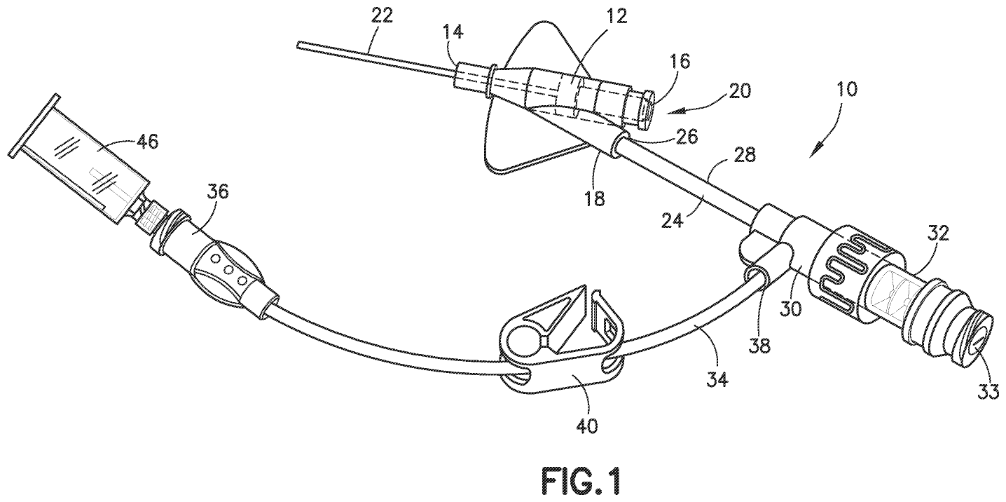

Referring now to FIG. 1, shown is a non-limiting embodiment of a catheter assembly 10 useful with blood draw devices as described herein below. Cather assembly 10 may include a catheter adapter 12, which may include a distal end 14 and a proximal end 16. In some embodiments, the catheter adapter 12 may include an additional port 18. In some embodiments, port 18 may disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. In some embodiments, the catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 25, a 60, or a 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removeably coupleable to catheter adapter 12, connector 30, and/or needleless access connector 32) including a second fluid conduit, such as second fluid conduit 34, that is coupled to a port 38 of the connector 30. Extension sets are known to those of skill in the art and are commercially available from, for example, Becton, Dickinson and Company. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

In some embodiments, the luer connection 36 at the proximal end of the second fluid conduit 34 may be coupled to one or more devices to effectuate blood draw, such as a luer lock access device (LLAD), e.g., LLAD 46. LLAD 46 may receive an evacuated container for withdrawing blood through second fluid conduit 34. In non-limiting embodiments, a syringe may be used to withdraw blood, by connecting such a syringe to luer connector 36 (e.g., without the need for LLAD 46).

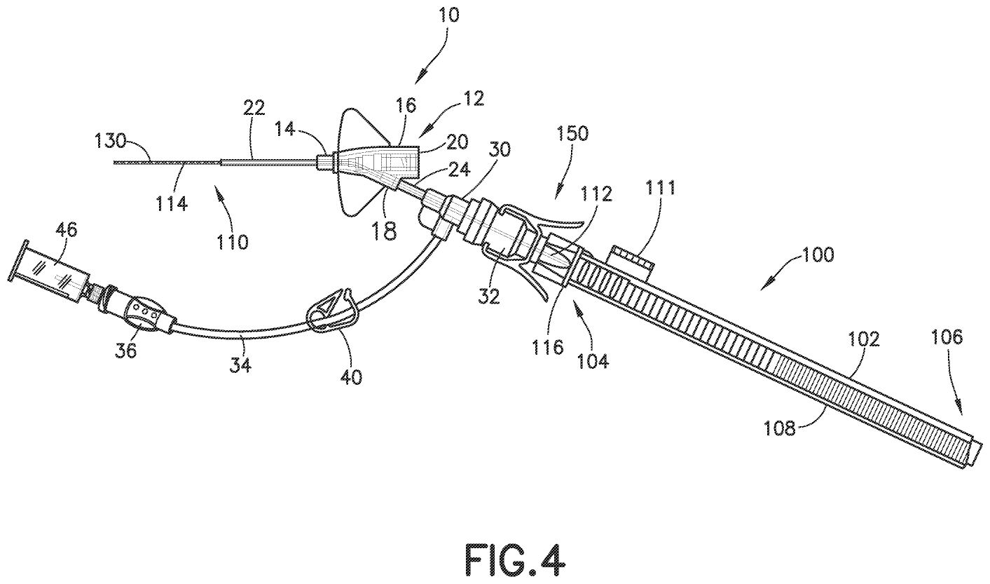

Catheter assembly 10 may include a needleless access connector 32 and/or a second fluid conduit 34. Suitable needleless access connectors 32 can include any split-septum connector, and/or those with direct fluid path access. Needleless access connectors 32 are known to those of skill in the art and are commercially available from, for example, Becton, Dickinson and Company under the tradenames MAXPLUS, MAXZERO, Q-SYTE, and SMARTSITE. Another example of a needleless access connector is the NSYTE Needle-Free Connector manufactured by NP Medical. While the non-limiting embodiments of FIGS. 1 and 4 show needleless access connectors arranged at connector 30, those of skill in the art will appreciate that suitable needleless access connectors may also be arranged at luer 36. In non-limiting embodiments, needleless access connector 32 includes a septum 33, such as a self-healing septum. In non-limiting embodiments, the septum 33 is a slit-type septum. As will be described below, a blood draw device as described herein may be reversibly coupleable to the needleless access connector 32, and one or more portions of the blood draw device may pierce the septum 33.

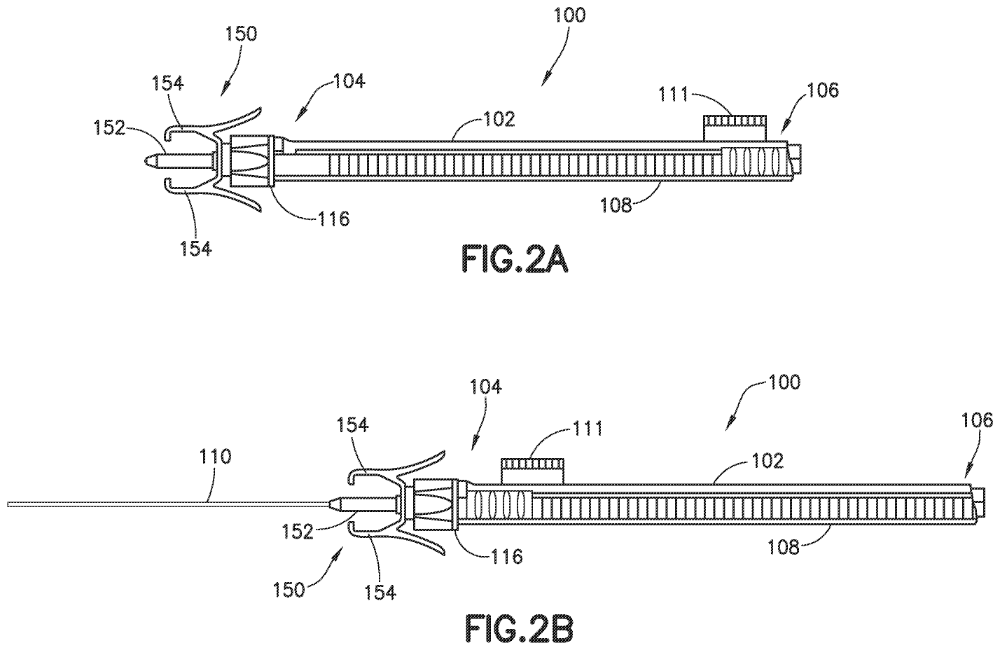

Turning to FIGS. 2A-2B, shown are non-limiting embodiments of an instrument delivery device 100 for introducing an instrument. As noted above, while the instrument delivery device 100 is described in the context of aiding in performing a blood draw via the catheter assembly 10 and associated instruments (e.g., extension set 34, 36, 40 and LLAD 46), those of skill will appreciate that application of antithrombogenic coatings to portions of an instrument employed in other environments, such as a probe (including an associated sensor) employed for prolonged diagnostic testing, accomplish a goal of the present invention, including reducing/preventing formation of blood clots on or in proximity to the probe. Exemplary instrument delivery and/or blood draw devices are described in, for example, U.S. Patent Application Publication No. 2020/0316346 and U.S. Pat. No. 10,300,247, the contents of which are incorporated herein by reference in their entirety.

Instrument delivery device 100 may include a housing 102 having a distal end 104, a proximal end 106, and a sidewall 108 that defines an interior volume of the housing 102. At the distal end 104 of housing 102, one or more structures are provided for reversibly coupling the housing 102 to catheter assembly 10, for example via needleless access connector 32 as shown in FIG. 4. Instrument delivery device 100 may include, in non-limiting embodiments, at distal end of housing 102, a lock 150, including one or more arm(s) 154 and a blunted cannula 152, for coupling to one or more portions of catheter assembly 10. Lock 150 may be any suitable size and/or shape, and may be formed of any suitable material(s), such as plastic(s). While lock 150 is exemplified in the present figures as a plurality of arms 154 and blunted cannula 152, those of skill will appreciate that any connection or coupling, for example a luer, can be used, so long as distal end of housing 102 of instrument delivery device 100 may pass through the connection to access catheter assembly 10.

Instrument delivery device 100 includes an instrument 110 received at least partially within the housing 102 of the device 100. In non-limiting embodiments, instrument 110 is a guidewire or probe received within instrument delivery device 100 and movable within an interior volume thereof, such as via movement of an advancement member 111 along the housing 102 (e.g., sliding within a slot (not shown) formed in the housing 102). The advancement member 111 extends into the interior volume of the housing 102 and is coupled to the instrument 110, such that movement of the advancement member 111 (in the proximal or distal direction) causes a corresponding movement of the instrument 110. When device 100 is coupled to catheter assembly 10, advancement member 111 may be moved distally so that instrument 110 advances through catheter adapter 12 and catheter 22, to enter a patient's vasculature. In non-limiting embodiments, instrument 110 is moveable within blunted cannula 152, such that while blunted cannula 152 enters a connector on catheter assembly 10, such as needleless access connector 32, instrument 110 may move relative to blunted cannula 152 to access catheter assembly 10. As shown in FIG. 4, instrument 110 may include a distal portion 114 and a proximal portion 112.

In some embodiments where instrument delivery device 100 is used in a blood draw procedure, blood may flow proximally from the catheter 22 to the catheter adapter 12 to the fluid conduit 24 to the second fluid conduit 34. In some embodiments, blood may be prevented from entering the delivery device 100 by a septum 116 that is positioned at the distal end 104 of the housing 102.

Instrument delivery device 100 may include one or more antithrombogenic coatings or compositions applied onto and/or integrated with components thereof, i.e., an “antithrombogenic material.” Specifically, and according to embodiments, the antithrombogenic material can be applied on or integrated into any region of instrument 110, for example, on the distal portion 114 (i.e., the portion that enters the patient's vasculature) or along the length of instrument 110. Suitable antithrombogenic and/or anticoagulant materials for use in medical devices that access the vasculature are known to those of skill in the art. The terms “antithrombogenic” and “anticoagulant” are used interchangeably herein, and suitable materials can be applied to, for example in the form of a film and/or a coating, one or more surfaces of one or more components of instrument delivery device 100 and/or may be used in the manufacture of one or more components (e.g., antithrombogenic moieties may be included in polymeric materials used during the production of components) of instrument delivery device 100.

Suitable antithrombogenic materials include without limitation, heparin, heparin-mimetic materials, heparin, albumin, hydrophobic lubricants, fluorinated lubricants, compositions including fluoro-containing functional moieties, silicone-containing functional moieties, and/or poly(ethylene glycol) (PEG) functional moieties, antithrombogenic polymers (e.g., those containing one or both of poly(ethylene oxide) (PEO)/sulfate/sulfonate and poly(propylene oxide) (PPO), poly(2-methoxyethyl acrylate)), and combinations, phosphorylcholine, pro-drugs, and derivatives thereof. Suitable antithrombogenic compositions are also available commercially, for example the Astute® Antithrombogenic Coating sold by BioInteractions Ltd. (Reading, UK). In non-limiting embodiments, the antithrombogenic composition includes heparin, PEG, and one or more sulfate and/or sulfonate-containing compositions. In non-limiting embodiments, the antithrombogenic composition is a PEO-PPO-PEO triblock copolymer.

In non-limiting embodiments, an antithrombogenic composition may be included with any material used to form the one or more components of instrument delivery device 100. For example, an instrument 110 may be formed of a suitable material and one or more antithrombogenic compositions may be included as a moiety or additive therewith. In non-limiting embodiments, the instrument 110 may include fluoro or fluoro-containing functional moieties or may be impregnated with antibiotic or antiseptic such as minocycline-rifampicin or chlorhexidine-silver sulphadiazine.

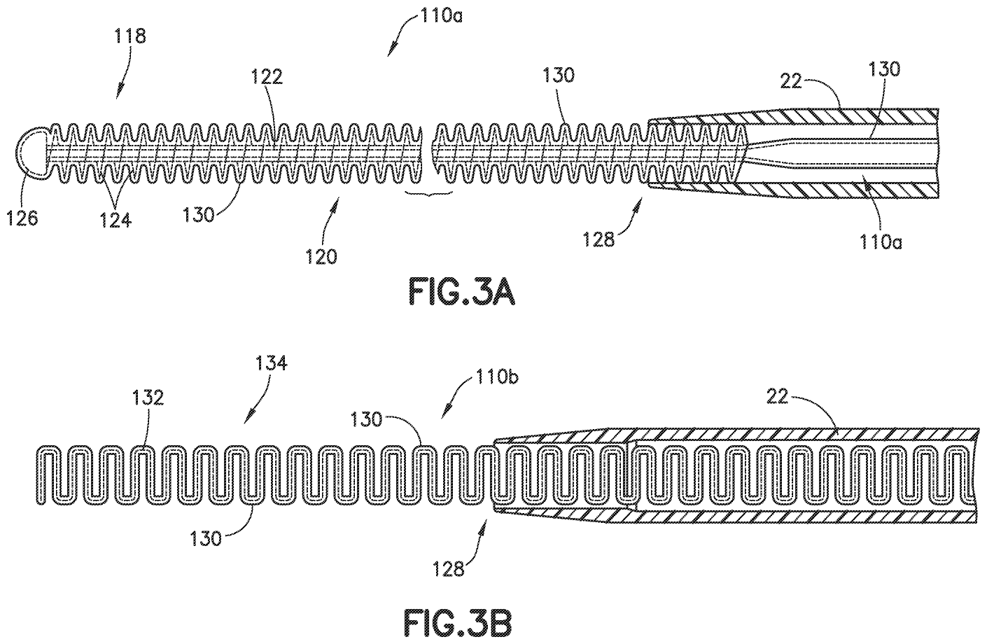

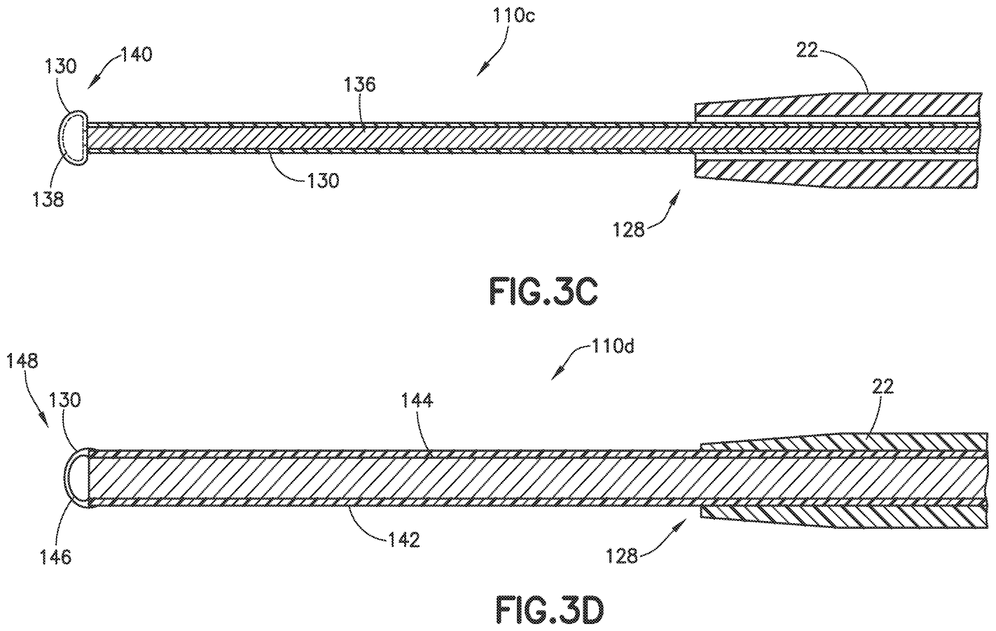

Referring now to FIGS. 3A-3D, example instruments 110 to which an antithrombogenic coating may be applied and/or with which an antithrombogenic composition may be integrated are shown in more detail. While the embodiments of FIGS. 3A-3D are illustrated with the instruments 110 having an antithrombogenic coating applied thereto, it is recognized that the instruments 110 could alternatively be manufactured of materials/compositions including antithrombogenic moieties therein, as described in detail previously, and thus the illustrated embodiments are understood to not limit the scope of the disclosure in this regard.

As first shown in FIG. 3A, an instrument 110 is provided in the form of a guidewire 110a, in accordance with one embodiment. The distal end 118 of the guidewire 110a includes a fluid permeable structure 120 thereon that is formed by an elongated core wire 122 and a coil 124 disposed in a helix about the elongated core wire 122. The fluid permeable structure 120 allows for blood to flow within a space between the elongated core wire 122 and the coil 124 in response to the guidewire 110a being inserted into the vasculature of a patient.

The elongated core wire 122 may be solid and/or constructed of metal (e.g., nitinol) or other suitable material, with the core 122 being thin to provide some flexibility. In some embodiments, the guidewire 110a may include a rounded distal tip 126, which may reduce a risk of damage to the vasculature when the guidewire 110a is inserted into the vasculature. In some embodiments, the rounded distal tip 126 may reduce a risk of thrombus development or other complications. In some embodiments, the coil 124 may be coupled to the elongated core wire 122.

The fluid permeable structure 120 of guidewire 110a may be advanced beyond the distal tip 128 of the catheter 22, which may move or push away anything within the vasculature of the patient that might otherwise occlude the catheter 22 during a blood draw. For example, the guidewire 110a may move, push away, or move beyond fibrin material or thrombosis, or move the distal tip 128 of the catheter 22 away from a vein wall or a valve.

As shown in FIG. 3A, the fluid permeable structure 120, i.e., elongated core wire 122 and coil 124, is coated with an antithrombogenic coating 130. The antithrombogenic coating 130 may have a composition according to any of the embodiments or combinations described in detail above. In some non-limiting embodiments, the antithrombogenic coating 130 may be included in a matrix (e.g., a cross-linked or polymerized coating) that allows for the antithrombogenic coating to be released, e.g., by virtue of being contacted with a liquid (e.g., blood) or by being exposed to one or more enzymes (e.g., in blood), as the blood draw device is received within catheter assembly 10. Suitable matrices for release, e.g., controlled release, of compositions are known to those of skill in the art and may include those disclosed in U.S. Pat. Nos. 8,821,455, 8,691,887, and 8,512,294, and U.S. Patent Application Publication Nos. 2013/0255061, 2010/0135949, and 2010/0136209, the contents of which are incorporated herein by reference in their entirety. In some embodiments, and as shown in phantom in FIG. 3A, an antithrombogenic coating 130 may also be applied to a remaining portion of the guidewire 110a extending proximally from fluid permeable structure 120.

Referring now to FIG. 3B, an instrument 110 is provided in the form of a guidewire 110b, in accordance with another embodiment. The guidewire 110b is constructed to have a fluid permeable structure that is formed by a coreless coil 132. The coreless coil 132 allows for blood to flow within a space between adjacent coils thereof in response to the guidewire 110b being inserted into the vasculature of a patient. The coreless coil 132 may be solid and/or constructed of metal (e.g., nitinol) or other suitable material, with the coils thereof being thin to provide some flexibility. The coreless coil 132 of guidewire 110b may be advanced beyond the distal tip 128 of the catheter 22, which may move or push away anything within the vasculature of the patient that might otherwise occlude the catheter 22 during a blood draw. For example, the coreless coil 132 of guidewire 110b may move, push away, or move beyond fibrin material or thrombosis, or move the distal tip 128 of the catheter 22 away from a vein wall or a valve.

According to embodiments, a distal end 134 (that extends out from catheter 22 when fully advanced) of the coreless coil 132 of guidewire 110b is coated with an antithrombogenic coating 130. As described above with regard to the guidewire 110a of FIG. 3A, the antithrombogenic coating 130 may be included in a matrix that allows for the antithrombogenic coating to be released upon being exposed to one or more enzymes in the blood, as the guidewire 110b is received within catheter assembly 10. In some embodiments, and as shown in phantom in FIG. 3B, an antithrombogenic coating 130 may also be applied to a remaining portion of the guidewire 110b contained within the catheter 22.

Referring now to FIG. 3C, an instrument 110 is provided in the form of a probe 110c, in accordance with another embodiment. The probe 110c generally includes a support wire 136 and a sensor 138 that is coupled to a distal tip 140 of the support wire 136. The support wire 136 may constructed of metal (e.g., nitinol) or other suitable material and is formed to have a thin profile such that the wire has some flexibility. The sensor 138 is configured to monitor various vital signs or physiological parameters of a patient, such as pressure, temperature, oxygen level, etc., and may be configured to wirelessly transmit measurements of such parameters to a remote receiver.

According to embodiments, each of the support wire 136 and sensor 138 is coated with an antithrombogenic coating 130. As described above with regard to the probe 110c of FIG. 3A, the antithrombogenic coating 130 may be included in a matrix that allows for the antithrombogenic coating to be released upon being exposed to one or more enzymes in the blood, as the probe 110c is received within catheter assembly 10. The antithrombogenic coating 130 acts to prevent blood clots from building on the surface of the probe 110c, and specifically the sensor 138, as over time such blood clots detrimentally affect performance of the sensor 138. Application of the antithrombogenic coating 130 to the sensor 138 can thus extend the useful life of the probe 110c.

Referring now to FIG. 3D, an instrument 110 is provided in the form of a probe 110d, in accordance with another embodiment. The probe 110d generally includes a protective outer tubing 142, an optical fiber 144 housed within the outer tubing 142, and an optical sensor 146 that is coupled to a distal tip 148 of the optical fiber 146 and tubing 142. The outer tubing 142 and optical fiber 144 form a flexible member that provides for bending of the probe 110d as it is inserted into the vasculature of a patient through catheter 22. The sensor 146 is configured to acquire measurements of vital signs or other physiological parameters as part of a diagnostic test and/or over a prolonged, continuous period of time, and may be configured to wirelessly transmit measurements of such parameters to a remote receiver.

According to embodiments, the distal tip 148 of the outer tubing 142 and the sensor 146 are coated with an antithrombogenic coating 130. As described above with regard to the probe 110a of FIG. 4A, the antithrombogenic coating 130 may be included in a matrix that allows for the antithrombogenic coating 130 to be released upon being exposed to one or more enzymes in the blood, as the probe 110d is received within catheter assembly 10. The antithrombogenic coating 130 acts to prevent blood clots from building on the surface of the probe 110d, and specifically the sensor 146, as over time such blood clots detrimentally affect performance of the sensor 146. Application of the antithrombogenic coating to the sensor 146 can thus improve diagnostic or continuous measurement capability of the sensor 146 (by preventing thrombus attachment or fouling of the sensor 146) and extend the useful life of the probe 110d.

Turning now to FIG. 4, shown is a system including a catheter assembly 10 as described above, and the instrument delivery device 100 as also described above. In the illustrated non-limiting embodiment, instrument delivery device 100, through lock 150, is coupled, optionally reversibly, to needleless access connector 32 of catheter assembly 10. With reference to the non-limiting embodiments of FIGS. 3A-3D, at least a distal portion 114 of the instrument 110 (i.e., a guidewire or probe) that extends out past catheter 22 (when advanced to that position by device 100) may be coated with an antithrombogenic coating 130. When instrument 110 is advanced into a portion of the catheter assembly 10, e.g. needleless access connector 32, distal portion 114 may be advanced through and beyond catheter 22 and into the vasculature of the patient. By virtue of inclusion of antithrombogenic coating 130 as a coating on one or more surfaces of distal portion 114, thrombus formation may be inhibited, allowing for blood draws to proceed without blockage caused by thrombogenesis, and without development of a thrombus in the patient's vasculature.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An instrument delivery device for advancement of an instrument into an intravenous catheter assembly, the instrument delivery device comprising:
a housing comprising a proximal end, a distal end, and a sidewall therebetween defining an interior, the housing comprising a connecter at the distal end thereof for reversibly connecting the instrument delivery device to the intravenous catheter assembly;
a displaceable instrument is received within the housing interior and includes a proximal end and a distal end, the instrument comprising one of a guidewire and a probe, wherein the instrument is advanceable from a first, proximal position in which the distal end does not extend beyond a distal end of a catheter of the intravenous catheter assembly to a second, distal position in which the distal end extends beyond the distal end of the catheter of the intravenous catheter assembly;
wherein the distal end of the instrument includes an antithrombogenic material, the antithrombogenic material comprising an antithrombogenic coating applied to at least part of the instrument or an antithrombogenic composition from which at least part of the instrument is formed.

2. The instrument delivery device of claim 1, wherein when the antithrombogenic material comprises the antithrombogenic coating, the antithrombogenic coating includes one or more of a hydrophilic material, heparin, a heparin mimetic, albumin, a hydrophilic lubricant, a fluorinated lubricant, and a triblock polymer.

3. The instrument delivery device of claim 2, wherein the triblock polymer comprises poly(ethylene oxide) (PEO) and poly(propylene oxide) (PPO).

4. The instrument delivery device of claim 3, wherein the triblock polymer is a PEO-PPO-PEO polymer.

5. The instrument delivery device of claim 2, wherein when the antithrombogenic material comprises the antithrombogenic coating, the antithrombogenic coating includes poly(ethylene glycol) or poly(ethylene oxide), one or more sulfate and/or sulfonate groups, and heparin.

6. The instrument delivery device of claim 1, wherein when the antithrombogenic material comprises the antithrombogenic coating, the antithrombogenic coating includes any of phosphorylcholine, and poly(2-methoxyethyl acrylate).

7. The instrument delivery device of claim 1, wherein when the antithrombogenic material comprises the antithrombogenic composition, the antithrombogenic composition includes fluoro or fluorine-containing functional moieties.

8. The instrument delivery device of claim 1, wherein when the antithrombogenic material comprises the antithrombogenic composition, the antithrombogenic composition includes an antibiotic or antiseptic comprising minocycline-rifampicin or chlorhexidine-silver sulphadiazine.

9. The instrument delivery device of claim 1, wherein the instrument comprises a guidewire including:
a core wire; and
a coil member wrapped around the core wire, at a distal end of the core wire;
wherein the antithrombogenic material is provided in or on the distal end of the core wire and the coil member.

10. The instrument delivery device of claim 1, wherein the instrument comprises a coreless coiled guidewire, and wherein the antithrombogenic material is provided in or on a distal end of the coreless coiled guidewire.

11. The instrument delivery device of claim 1, wherein the instrument comprises a probe including:
a support wire; and
a sensor positioned at a distal end of the support wire configured to measure one or more physiological parameters;
wherein the antithrombogenic material is provided in or on the distal end of the support wire and applied to the sensor as an antithrombogenic coating.

12. The instrument delivery device of claim 1, wherein the instrument comprises a probe including:
an outer tubing;
an optical fiber contained within the outer tubing; and
a sensor coupled to a distal end of the optical fiber, at a distal tip of the outer tubing, the sensor configured to measure one or more physiological parameters;
wherein the antithrombogenic material is provided in or on the distal tip of the outer tubing and applied to the sensor as an antithrombogenic coating.

13. The instrument delivery device claim 11, wherein the sensor is configured to acquire measurements of the one or more physiological parameters over a continuous period of time.

14. The instrument delivery device of claim 13, wherein application of the antithrombogenic coating to the sensor prevents fouling of the sensor.

15. The instrument delivery device of claim 1, wherein the antithrombogenic material is provided in or on an entire length of the instrument.

16. The instrument delivery device of claim 1, further comprising an advancement member positioned on the housing and moveable relative thereto, the advancement member coupled to the instrument such that movement of the advancement member causes a corresponding movement of the instrument.

17. The instrument delivery device of claim 1, wherein the connector comprises a plurality of arms configured to engage a needleless connector.

18. The instrument delivery device of claim 1, wherein the connector comprises a blunted cannula configured to pierce a septum of the needleless connector.

19. A catheter system, comprising:
a catheter assembly comprising:
a catheter adapter, comprising a distal end, a proximal end, a lumen extending between the distal end and the proximal end; and
a catheter secured to the catheter adapter and extending distally from the catheter adapter, wherein the catheter comprises a distal end and a proximal end; and
an instrument delivery device comprising:
a housing comprising a proximal end, a distal end, and a sidewall therebetween defining an interior, the housing comprising a connecter at the distal end thereof for reversibly connecting the instrument delivery device to the intravenous catheter assembly;
a displaceable instrument is received within the housing interior and includes a proximal end and a distal end, the instrument comprising one of a guidewire and a probe, wherein the instrument is advanceable from a first, proximal position in which the distal end does not extend beyond a distal end of a catheter of the intravenous catheter assembly to a second, distal position in which the distal end extends beyond the distal end of the catheter of the intravenous catheter assembly;
wherein the distal end of the instrument includes an antithrombogenic material, the antithrombogenic material comprising an antithrombogenic coating applied to at least part of the instrument or an antithrombogenic composition from which at least part of the instrument is formed.

20. The system of claim 19, wherein the catheter assembly comprises a needleless access connector, and wherein the distal end of the instrument delivery device housing is reversibly coupleable to the needleless access connector.
